(19) United States
(12) Patent Application Publication
(10) Pub. No.: US 2026/0216441 A1
(43) Pub. Date: Jul. 30, 2026

(54) PLUNGER ROD, SUB-ASSEMBLY AND MEDICAMENT DELIVERY DEVICE

(71) Applicant: SHL Medical AG, Zug (CH)

(72) Inventors: Amit Singh, Deerfield Beach, FL (US); Joakim Gunnar Lindholm, Nacka Strand (SE)

(21) Appl. No.: 19/148,132

(22) PCT Filed: Dec. 13, 2023
(86) PCT No.: PCT/EP2023/085576

§ 371 Date: Jul. 15, 2025

Related U.S. Application Data
(60) Provisional application No. 63/440,948, filed on Jan. 25, 2023.

(30) Foreign Application Priority Data
Feb. 22, 2023 (EP) 23158066.3

(51) Int. Cl. A61M 5/315; A61M 5/24; A61M 5/31

(52) U.S. Cl. CPC A61M 5/31511; A61M 5/24; A61M 2005/2418; A61M 2005/3143; A61M 2202/0007

(57) ABSTRACT

The present disclosure provides a plunger rod for a medicament delivery device, the plunger rod including a first section configured as shock absorber and a second section along a longitudinal axis of the plunger rod. A stiffness of the second section exceeds a stiffness of the first section by a factor of at least five two.

### TECHNICAL FIELD

The invention refers to a plunger rod for a medicament delivery device, to a sub-assembly for a medicament delivery device, and to a medicament delivery device.

### BACKGROUND

Medicament delivery devices, in short delivery devices, include pen type manual injectors and auto-injectors. These delivery devices are generally known for the self-administration of a medicament by patients without formal medical training. For example, patients suffering from diabetes may require repeated injections of insulin, or patients may require regular injections of other types of medicaments, such as a growth hormone.

A medicament delivery device includes a medicament container, in short container, for storing the medicament. The container comprises an outlet at its proximal end, e.g. a needle, and a stopper slidable within the container. The medicament is stored in the container between the outlet and the stopper. The stopper seals the medicament container and may be made from rubber, for example. In case a medicament delivery is desired, a plunger rod of the delivery device is activated and pushes the stopper further into the container thereby expelling a dose of the medicament at the outlet of the container.

A force evoked by the actuated plunger rod when hitting the stopper may in some scenarios be sufficiently high to cause damage at the medicament container and, in a worst case, even cause glass breakage. Excess plunger rod forces may result from variations in the plunger rod movement, from variations in the activation energy, from variations in material and/or geometry of a biasing element (for instance a drive spring guided by a guide rod) used to move the plunger rod, or from friction the plunger rod is exposed to during movement.

One specific scenario embraces a medicament container being only partly filled, e.g. when using the same medicament delivery device for various dosage levels. In such scenario, the stopper is arranged deeper in the medicament container compared to a fully filled scenario. Therefore, the plunger rod accelerates for a longer way and a longer time and the stopper is hit with increased kinetic energy.

### SUMMARY

Reference should now be made to the appended claims.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a longitudinal axis extending in the direction of the longest extension of the device and/or component. Similarly, “radial” or “radially” refer to a direction extending radially relative to the axis, and “rotation”, “rotational” and “rotationally” refer to rotation relative to the axis.

When a component is said to move proximally, distally, axially in a proximal direction, axially in a distal direction or equivalent terms, the movement is relative to the housing of the injection device, unless mentioned otherwise.

According to a first aspect of the present invention, a plunger rod is provided for a medicament delivery device. The plunger rod extends along a longitudinal axis between a proximal end and a distal end. The plunger rod comprises at least a first section and a second section extending along the longitudinal axis, preferably adjacent each other, preferably starting at the proximal end and aligned in distal direction, however not necessarily. The first section of the plunger rod is configured as shock absorbing element. A stiffness of the second section thereby exceeds a stiffness of the first section by a factor of at least two, preferably by a factor of at least five, preferably at least eight. Alternatively, or in addition to the stiffness measures, the first section is compressible along the longitudinal axis for at least 0.5 mm, preferably for at least 1 mm, without rupture.

The stiffness of a body or a section of a body is defined as κ=F/δ with F indicating the force acting in direction of the degree of freedom of the body, and δ being the displacement in that direction in response to the force F. Presently, the stiffness measures for the first and the second section refer to a degree of freedom along the longitudinal axis each. The ratio of the two stiffness measures indicates that the first section is embodied as shock absorbing element while the second section represents the original characteristic of a plunger rod being sufficiently stiff to transfer the kinetic energy of the actuated plunger rod to the stopper and causing the stopper to move in the medicament container.

The stiffness of a body or a section of a body is determined by its material and its geometry. One or both of these parameters of the first and the second section of the plunger rod are preferably tuned to meet the above ratio of stiffness measures and to meet the functionality of the first section as a shock absorbing element. This being said, the first section and the second section may be integral sections of the plunger rod, or, in an alternative, may be separate sections mounted to each other.

It is preferred that the first section of the plunger rod is designed to a stiffness, in absolute numbers, between 50 N/mm and 250 N/mm, preferably between 140 N/mm and 180 N/mm, more preferably between 155 N/mm and 165 N/mm, most preferably 160 N/mm. These ranges are determined in view of an average force required to compress the first section of the plunger rod preferably exceeds an activation force for the plunger rod, which activation force may stem from a plunger rod biasing element (for instance a drive spring guided by a guide rod) force as explained in more detail below. In the example of the stiffness being configured to ~160 N/mm, in case at least the first section being made from ABS, and in a sample application, this first section compresses for 1 mm. This corresponds to absorbing approximately a 20N actuation force flying a distance of 8 mm. It is assumed that in a pre-delivery state of the delivery device the plunger rod is located distant from the stopper, i.e. the above flying distance, so as to travel before hitting the stopper. The above stiffness ranges are considered as sufficient to absorb excess energy in the first section of the plunger rod. Accordingly, the second section of the plunger rod preferably is designed to a stiffness of 500 N/mm and more. In case of a very low stiffness of the first section within the above ranges, the stiffness of the second section may even be as low as 100 N/mm.

By way of providing a shock absorbing, i.e. a dampening or attenuating element in the plunger rod, excess plunger rod or powerpack force is absorbed by preferably an elastic deformation of the shock absorbing element. Accordingly, any excess force stemming from variations in the plunger rod movement, and/or from frictional forces the plunger rod is exposed during its movement may be attenuated to a level no longer causing damage to the medicament container, or to other components of the delivery device such as sensitive plastic parts.

In a preferred embodiment, the first section of the plunger rod terminates the plunger rod at a proximal end. Given that during operation the plunger rod meets or hits the stopper with its proximal end, the plunger rod preferably meets or hits the stopper with the shock absorbing element/section first. Specifically, there is no member of higher stiffness provided in proximal direction other than the shock absorbing element. In particular there is no engagement member of higher stiffness provided at the end of the first section to engage with the stopper during operation.

In an embodiment of the present invention, the first section of the plunger rod comprises a resilient member while the second section of the plunger rod comprises a tubular member configured to receive a biasing element (for instance a drive spring guided by a guide rod) extending along the longitudinal axis. The tubular member also comprises an integrated front at its proximal end for biasing the biasing element against, and an open, distal end for receiving the biasing element. The resilient member is elastic such that a compression of this member is reversible absent the impact of a force. Hence, resilient member can also be considered as springy member that absorbs at least some of the impact energy.

The biasing element may be a compression spring or a torsion spring which in response to triggering a release mechanism accelerates the plunger rod in proximal direction relative to a housing for the plunger rod. In an assembled delivery device, the plunger rod then hits and moves a stopper of a medicament container in proximal direction causing a dose of the medicament to be delivered.

In one embodiment of the present invention, the first section of the plunger rod is made from the same material as its second section. Preferably, it is only one material the first and the second section are made from in their entirety, i.e. no other material contributes to the first and second section, and preferably to the entire plunger rod. A material of choice can be a polymer or plastic material, such as one of ABS, PA, POM, PBT. In this embodiment, the different stiffness measures of the first and the second section are caused by different geometric structures. While the second section is shaped so as to provide a stiff skeleton to hold the plunger rod loaded while in storage, the first section is designed as shock absorbing element with considerable lower stiffness. This may be achieved by designing the first section as compressible tubular structure comprising a circumferential perforated shell. In this embodiment, an outer diameter of the first section and the second section preferably is the same. Even an inner diameter of the first and the second section may be the same. In other embodiments, however, the outer and/or inner diameters may vary. The first section is desirably designed stiff enough to absorb a significant amount of energy, and soft enough to so that an impact force is kept low.

In case the resilient member is represented by the perforated shell, such perforated shell preferably is arranged at the proximal end of the plunger rod, again absent any other plunger rod member of higher stiffness closer to the stopper in an assembled state. Preferably, perforations in the shell at adjacent levels along the longitudinal axis are arranged circumferential offset. This supports the shell to maintain its circumferential shape even under compression. In a preferred embodiment, two diamond or elliptic shaped perforations are arranged per level on the circumference of the shell. Centres of the perforations of adjacent levels are circumferential offset by a quarter of the circumference of the shell, and circumferential adjacent perforations in adjacent levels overlap. All these embodiments ensure that during envisaged compression of the shell, the shell maintains its circumferential shape also under compression such that no portions of the shell radially bend which may cause damage during operation, too.

Preferably, the first and the second section are integral sections of the plunger rod. This means, that the first and the second section are manufactured in common manufacturing steps, e.g. in an injection moulding process with a mould common for both sections/parts. In other words, in case of resilient and tubular member, the resilient member and the tubular member are not manufactured as separate pieces that need to be mounted to each other later on. Additional manufacturing steps can be avoided. Preferably, the resilient member protrudes from the tubular member at the proximal end, i.e. from the front integrated with the tubular member.

In a different embodiment, the first section and the second section of the plunger rod are separate members mounted to each other. This embodiment preferably is applied in case the different stiffness measures shall mainly be determined by the choice of material rather than geometry. Accordingly, two different materials are preferably used for realizing the first and the second section of the plunger rod. Accordingly, it is preferred that the first section is made from a material with a Young modulus lower than the Young modulus of a material the second section is made from. Preferably, the material of the first section has a Young modulus at least five times lower than the material of the second section. While the second section preferably represents the tubular member, this second section preferably is made from one of ABS, PA, POM, PBT. The material selected for the first section representing the resilient member preferably is one of rubber and foam. Any other elastic material can be used to build the first section from.

The resilient member in this embodiment preferably is a solid member. A solid member is defined as a member absent through holes or recesses. This facilitates manufacturing of the resilient member and mounting the resilient member to the tubular member. This resilient member serves as protection element, and preferably is of cylindrical shape. While the tubular member preferably is manufactured by injection moulding, the resilient member may be manufactured by one or more of cutting, pressing, foaming, etc.

In preferred embodiments, the resilient member is mounted to the front of the tubular member by one or more of gluing, foaming and one or more locking ribs protruding from a mounting face of the resilient member. For the latter mounting alternative, the front of the tubular member comprises one or more corresponding recesses configured to interact with the locking ribs and contributing to one of a snap fit or a press-fit in combination with the locking ribs. Of course, alternatively the locking ribs may be provided on the front of the tubular member and corresponding recesses may be provided in the resilient member.

According to another aspect of the present invention, a sub-assembly is provided for a medicament delivery device. The sub-assembly, i.e. a drive unit, comprises a housing and a plunger rod according to any of the preceding embodiments. The plunger rod is slidable arranged in the housing along the longitudinal axis. Accordingly, the sub-assembly is configured to trigger the output of a dose of a medicament from a medicament container of a delivery device, in an assembled state. For this purpose, the sub-assembly in addition comprises an activation element, also called biasing element, preferably a drive spring such as a compression spring or a torsion spring. The biasing element preferably is arranged in the tubular member of the plunger rod and is biased against the integrated front of the tubular member at its proximal end and against the housing at the distal end. The distal end of the housing may be represented by a cap, while the portion of the housing enclosing the plunger rod may be of tubular shape and is also referred to as plunger guide. In the pre-tensioned state of the biasing element, mechanical potential energy is stored in the sub-assembly. This reflects a pre-delivery state of the corresponding delivery device. The sub-assembly preferably comprises a release mechanism configured to release the plunger rod thereby converting the potential energy stored in the biasing element into kinetic energy of the plunger rod.

According to a further aspect of the present invention, a medicament delivery device is provided. The delivery device comprises a medicament container configured to store a medicament. The container extends along the longitudinal axis between a proximal end and a distal end, and in one embodiment is a syringe. The container comprises an outlet at its proximal end. The outlet may comprise one or more of an opening, a taper of the container, a needle, a needle assembly. A stopper is arranged in the container and is configured to prevent leakage of the medicament from the distal end of the container. In case the container is filled to a maximum by the medicament, the stopper is arranged at the distal end in the container, e.g. in a press-fit. In case the container has a tubular shape, the stopper preferably is disk- or cylindrical-shaped and has a diameter equal to the inner diameter of the container. The stopper is arranged slidable in the medicament container along the longitudinal axis. In response to such movement of the stopper towards the proximal end, the medicament is delivered through the outlet. The delivery device further comprises a shock absorbing element configured to attenuate a shock resulting from the plunger rod hitting the stopper in response to an actuation of the plunger rod. The shock absorbing element is either embodied in the plunger rod which is a plunger rod according to any of the preceding embodiments. As laid out above, the shock absorbing element may be represented by a resilient member of the plunger rod, embodied as section integrated in the plunger rod, or embodied as separate first member attached to a second member of the plunger rod.

Or, the shock absorbing element is embodied as a resilient member mounted to or integrated with the stopper. In case of a separate resilient member to be mounted to the stopper, the resilient member may be embodied in the same way as in the related embodiments above. For example, the resilient member is attached to a distal end of the stopper by one or more of gluing, foaming, and one or more locking ribs protruding from a face of the resilient member that reaches into one or more corresponding recesses in the stopper and, together, contribute to one of a snap fit or a press-fit.

All other preferred properties-such as choice of material, elasticity, etc.-of the resilient member mounted to the plunger rod as described above are also applicable to the resilient member mounted to the stopper.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings, wherein

FIG. 1 illustrates a side view on a plunger rod according to an embodiment of the present invention;

FIG. 2 illustrates a lateral cut view of a portion of a medicament delivery device, except for a plunger rod in side view, according to embodiment of the present invention;

FIG. 3 illustrates a side view of a resilient member of a plunger rod according to embodiment of the present invention, in an extended state in diagram a), and in a compressed state in diagram b);

FIG. 4 illustrates a perspective view on a shock absorbing element as used in any of a plunger rod, a sub-assembly and a medicament delivery device according to the present invention;

FIG. 5 illustrates a lateral cut view of a portion of a medicament delivery device according to an embodiment of the present invention;

FIG. 6 illustrates a lateral cut view of a portion of a medicament delivery device according to another embodiment of the present invention; and

FIG. 7 illustrates a perspective transparent view of a sub-assembly according to an embodiment of the present invention.

### DETAILED DESCRIPTION

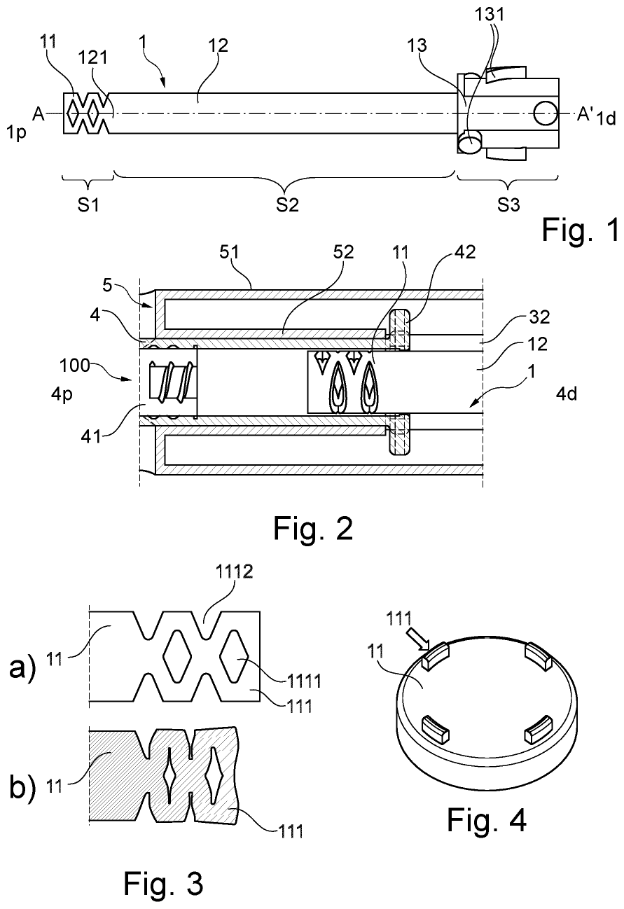

FIG. 1 illustrates a side view of a plunger rod 1 according to an embodiment of the present invention. The plunger rod 1 extends along a longitudinal axis A-A′ between a proximal end 1p and a distal end 1d. The plunger rod 1 is intended for use in a medicament delivery device (not shown), preferably in an auto-injector. In such application, the plunger rod 1 is expected to hit and subsequently push a stopper of a medicament container arranged in the auto-injector, such as a syringe. The syringe extends along the longitudinal axis between a proximal end comprising an outlet for a medicament stored in the container, and a distal end. The stopper is arranged at a position between the proximal end and the distal end of the container and prevents leakage of the medicament at the distal end. The stopper is arranged slidable in the container, such that in response to the plunger rod hitting the stopper the stopper is pushed within the medicament container from its actual position, e.g. at the distal end, towards the proximal end of the container, thereby delivering a dose of the medicament at the outlet to a patient. The outlet may comprise one or more of an opening in the container, a tapering of the container, a needle, a needle assembly, etc.

The plunger rod 1 comprises, along the longitudinal axis, a first section S1 represented by resilient member 11 at its proximal end 1p, a second section S2 represented by a tubular member 12, and a third section represented by an interface member 13 at its distal end 1d. The tubular member 12 is configured to receive a biasing element, here a drive spring guided by a guide rod, both extending along the longitudinal axis A-A′. The interface member 13 is tubular shaped as well and has a through hole along the longitudinal axis for receiving the drive spring and the guide rod. Accordingly, the interface member 13 has an opening at its distal end id allowing to introduce the drive spring and the guide rod into the tubular member 12. The tubular member 12 comprises a front 121 at its proximal end closing the tubular member 12, which front 121 serves as a bias for the drive spring. In a corresponding auto-injector, the drive spring is compressed, i.e. pre-tensioned in the pre-delivery state of the auto-injector. The drive spring, hence, is biased between the front 12 of the plunger rod 1 and a housing enclosing the plunger rod 1. The housing preferably comprises a front facing the interface member 13 of the plunger rod 1, and an outer shell enclosing the plunger rod, wherein the plunger rod 1 is preferably slidable mounted in the housing against the force of the drive spring. The interface member 13 comprises one or more protrusions 131 in form of noses or guides that interact with corresponding recesses e.g. in a shell of the housing, for implementing a release mechanism. A diameter and circumference of the interface member 13 is aligned with an inner diameter and circumference of the shell of the housing. On the other hand, the diameter of the interface member 13 exceeds a diameter of the tubular member 12 such that the transition from the interface member 13 to the tubular member 12 may, in one embodiment, act as a stop for a translational movement of the plunger 1.

The resilient member 11 protrudes from the tubular member 11 at the proximal end 1p and is configured as compressible hollow structure comprising a perforated shell allowing a compression along the longitudinal axis. FIG. 3 illustrates a side view of such resilient member 11 of a plunger rod in an extended state in diagram a) absent any force acting in longitudinal direction, and in a compressed state in diagram b) under a compression force evoked e.g. by the plunger rod 1. As can be derived, the resilient member 11 is compressed in axial direction and serves as shock absorbing element that converts a portion of the kinetic energy into deformation/thermal/frictional energy. This deformation attenuates the impact of the plunger rod onto the stopper and prevents damaging the container or other structural interfaces of the delivery device in response to the impact, such as snap fits that hold a front sub-assembly and a rear sub-assembly of the delivery device together.

The resilient member 11 of FIG. 3 is represented by a shell 111 comprising perforations 111, 1112 along its circumference as well as different levels on the longitudinal axis. The perforations 1111,1112 that are arranged at adjacent levels of the longitudinal axis are arranged circumferential offset. Specifically, two diamond or elliptic shaped perforations 1111 are arranged per level on the circumference of the shell 111 while centres of perforations 1111,1112 of adjacent levels are circumferential offset by a quarter of the circumference of the shell 111. Perforations 1111,1112 in adjacent levels overlap.

Returning to FIG. 1, the resilient member 11, the tubular member 12 and the interface member 13 are all integral members of the plunger rod 1. The plunger rod 1 preferably is made from one of ABS, PA, POM, PBT, and preferably is manufactured as one piece by injection moulding.

Accordingly, the present plunger rod 1 has, along its longitudinal axis, at least two sections of different stiffness, i.e. a first section S1 represented by the resilient member 11, and a second section S2 presently represented by the tubular member 12, wherein the stiffness of the second section S2 exceeds the stiffness of the first section S1 by at least a factor of five.

FIG. 2 illustrates a lateral cut view of a portion of a medicament delivery device 100 including a plunger rod 1 in side view, according to embodiment of the present invention. The present cut-out of the medicament delivery device 100 includes a medicament container 4, preferably of tubular shape, e.g. a syringe. A stopper 41 is arranged in the container 4, e.g. of cylindrical or plug shape. Accordingly, the medicament is provided in the container 4 in proximal direction from the stopper 41 (not shown). The stopper 41 seals the container 4 such that no medicament can leak from the container 4 in distal direction.

The container 4 is supported by a container carrier 5. The container carrier 5 also is of tubular shape such that the container 4 can be received in the interior of the container carrier 5. The container carrier 5 comprises a two-wall set-up with an outer wall 51 and an inner wall 52. The inner wall 52 does not extend all along the outer wall 51 along the longitudinal extension in distal direction. At a distal end 4d, the container 4 has a radial protrusion 42 that serves as a stop for the container 4 being inserted into the container carrier 5 in proximal direction. A plunger rod 1, e.g. the plunger rod of FIG. 1, is slidable mounted in a plunger guide 32 which plunger guide 32 may be a part of the housing of a sub-assembly driving the plunger rod 1. The plunger guide 32 abuts against the protrusion 42 or against the inner wall 52 of the container support 5.

The operational state of the delivery device illustrated in FIG. 2 is prior to the plunger rod 1 hitting the stopper 2, i.e. during acceleration of the plunger rod in proximal direction. On the other hand, the stopper 42 is arranged distant from the distal end of the container 4 which may indicated that a volume of the container 4 is not completely filled by the medicament in view of a smaller dosage to be delivered. Hence, the plunger rod 1 is accelerated for an additional distance compared to a completely filled container 4, such that the impact of the plunger rod 1 on the stopper 41 may cause excess vibrations/shock that may be transferred to the container 4 via the stopper 41.

The resilient member 11 of the plunger rod 1 dampens the impact of the accelerated plunger rod 1 on the stopper 41 while still transferring a sufficient force on the stopper 41 to move the stopper 41 towards the proximal end for medicament dosage delivery.

FIG. 4 illustrates a perspective view of a shock absorbing element as used in any of a plunger rod, a sub-assembly and a medicament delivery device according to the present invention. This shock absorbing element is also referred to as resilient member 11 and presently has the shape of a solid disk configured to attenuate shocks and/or vibrations. The resilient member 11 comprises protrusions in form of locking ribs 111 on its face that is the mounting face of the resilient member 11. The locking ribs 111 are configured to interact with corresponding recesses in the respective face of a member the resilient member 11 is to be mounted to which element can either be a stopper or a member of a plunger rod.

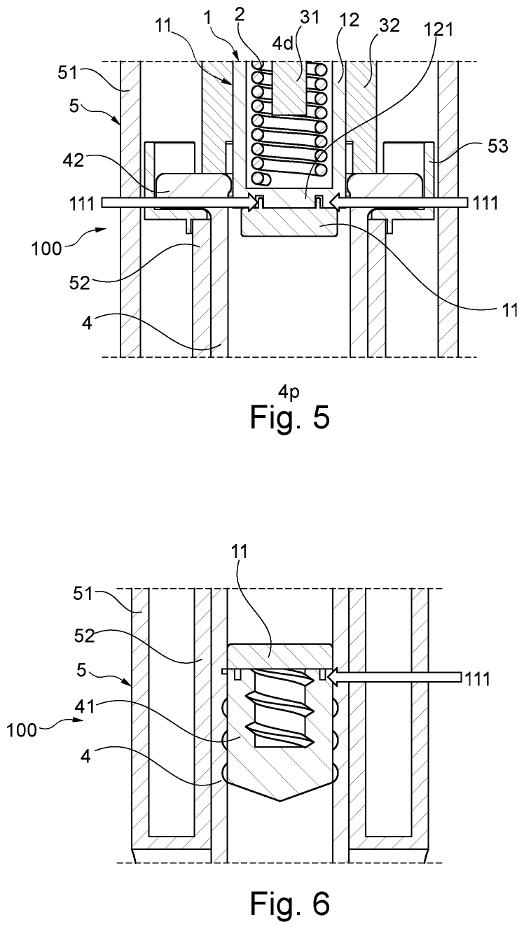

FIG. 5 illustrates a lateral cut view of a portion of a medicament delivery device 100 according to an embodiment of the present invention. The present portion of the medicament delivery device 100 shows a medicament container 4. At the distal end 4d of the container 4, the container 4 provides a radial protrusion 42, and the container 4 represents a syringe with a needle at the proximal end 4p (not shown). A container carrier 5 has an inner wall 52 and an outer wall 51, the inner wall 52 being shortened in distal direction with respect to the outer wall 51. At this distal end 4d of the inner wall 52, an insert 53 is provided that is adapted to the geometry of the syringe and in particular of its radial protrusion 43. During assembly of the delivery device 100, the syringe is inserted into the container carrier 5 at its distal end 4d until the protrusion 43 meets the insert 53. During assembling a sub-assembly comprising a plunger rod 1 and a housing 3 to the container carrier 5, the housing 3 is stopped by the protrusion 42.

In the situation illustrated in FIG. 5, the stopper of the syringe is not shown, i.e. the stopper is not arranged at the distal end 4d of the container 4 but is expected to be located further down in the container 4 towards the proximal end 4p. Again, this may indicate a scenario, in which the syringe is only partly filled with the medicament.

In this embodiment of a delivery device 100, the resilient member 11, which preferably is the resilient member 11 shown in FIG. 4, is mounted to a front 121 of a tubular member 11 of the plunger rod 1.

FIG. 6 illustrates a lateral cut view of a portion of a medicament delivery device according to another embodiment of the present invention. Again, a medicament container 41 is held by a container carrier 5 with an inner wall 52 and an outer wall 51. In comparison with the embodiment of FIG. 5, the plunger rod no longer is shown, but a stopper 41 of the container 4 is. In this embodiment, the resilient member 11 no longer is mounted to a member of the plunger rod 1, but is mounted to a distal end of the stopper 41, which is the end facing the plunger rod. As to the properties and the mounting of the resilient member 11 it is referred to the description of FIGS. 4 and 5.

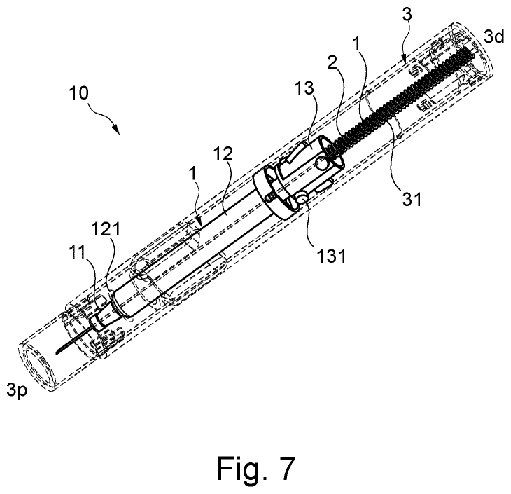

FIG. 7 illustrates a perspective and partially transparent view of a sub-assembly according to an embodiment of the present invention. The sub-assembly includes a tubular housing 3 with a closed distal end 3d and an open proximal end 3p. Inside the housing, a plunger rod 1, e.g. the plunger rod 1 of FIG. 1, is slidable arranged along the longitudinal axis. The plunger rod 1 is illustrated in an extended state, e.g. after a drive spring 2, that is guided by a guide rod 31 of the housing 3, has effected an acceleration of the plunger rod 1 towards the proximal end 3p. Hence, the drive spring 2 is shown in a relaxed, i.e. non-pre-tensioned state. Previously, the drive spring 2 was in a pre-tensioned state, biased against a front 121 of the plunger rod 1 and the closed distal end 3d of the housing 3. It should be noted that the drive spring and guide rod may be replaced with another kind of biasing element able to accumulate energy and moving the plunger rod when releasing it.

The housing 3 and the interface member 13 of the plunger rod 1 implement a release mechanism by means of the protrusions 131 of the interface member 13 and guides indicated in the housing 3. For example, by means of its protrusions 131, the plunger rod 1 may be rotated and locked in a lock position, in which the drive spring is held in the pre-tensioned state. The release mechanism may include means to release this lock position of the plunger rod 1.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

1-15. (canceled)

16. A plunger rod for a medicament delivery device, the plunger rod comprising:
a first section configured as shock absorbing element and a second section,
wherein each of the first section and the second section are positioned along a longitudinal axis of the plunger rod between a proximal end of the plunger rod and a distal end of the plunger rod, and
wherein a stiffness of the second section exceeds a stiffness of the first section by a factor of at least two.

17. The plunger rod of claim 16, wherein the first section is compressible along the longitudinal axis for at least 0.5 mm without rupture.

18. The plunger rod of claim 16,
wherein the stiffness of the first and second section each refer to a degree of freedom along the longitudinal axis, and
wherein the first section is configured to a stiffness between 50 N/mm and 250 N/mm, preferably between 140 N/mm and 180 N/mm, more preferably between 155 N/mm and 165 N/mm.

19. The plunger rod claim 16,
wherein the first section terminates the plunger rod at the proximal end absent any engagement member of lower stiffness in proximal direction,
wherein the second section succeeds the first section in distal direction.

20. The plunger rod of claim 16,
wherein the first section of the plunger rod is represented by a resilient member,
wherein the second section of the plunger rod comprises a tubular member configured to receive a biasing element extending along the longitudinal axis, and
wherein the tubular member comprises an integrated front at its proximal end for biasing the biasing element against.

21. The plunger rod of claim 16,
wherein the first and the second section of the plunger rod are integral members and are made from the same material, preferably which material is a polymer or a plastic material, and more preferably which material is one of ABS, PA, POM and PBT.

22. The plunger rod of claim 21,
wherein the resilient member is represented by a tubular structure compressible along the longitudinal axis, the tubular structure comprising a circumferential perforated shell arranged at the proximal end of the plunger rod.

23. The plunger rod of claim 15,
wherein perforations arranged in the shell at adjacent levels along the longitudinal axis are arranged circumferential offset,
preferably wherein two diamond or elliptic shaped perforations are arranged per level along the circumference of the shell, wherein centres of the perforations of adjacent levels are circumferential offset by a quarter of the circumference of the shell, and wherein perforations in adjacent levels overlap.

24. The plunger rod of claim 16,
wherein the first section and the second section of the plunger rod are mounted to each other.

25. The plunger rod of claim 24,
wherein the first section is made from a material with a Young modulus lower than the Young modulus of a material of the second section.

26. The plunger rod of claim 25,
wherein the resilient member is a solid member and is made from one or more of rubber and foam.

27. The plunger rod of claim 26,
wherein the resilient member is mounted to the front by one or more of:
gluing;
foaming;
one or more locking ribs protruding from a face of the resilient member inserted into corresponding recesses in the front of the tubular member in one of a snap fit or a press-fit.

28. A sub-assembly for a medicament delivery device, the sub-assembly comprising:
a housing; and
a plunger rod according to claim 16 arranged slidable in the housing along the longitudinal axis.

29. The sub-assembly of claim 28, further comprising:
a biasing element arranged in the tubular member of the plunger rod and, at least in a pre-tensioned state, biased against the front of the tubular member and against the housing; and
a release mechanism configured to release the biasing element from its pre-tensioned state for accelerating the plunger rod in proximal direction.

30. A medicament delivery device, comprising:
the plunger rod of claim 16;
a medicament container configured to store a medicament, the medicament container extending along a longitudinal axis between an outlet at a proximal end and a distal end;
a stopper arranged slidable in the medicament container along the longitudinal axis, wherein the stopper is configured to prevent leakage of the medicament from the distal end (4d) of the medicament container, and wherein the stopper is configured to push the medicament through the outlet in response to an actuation of the plunger rod; and
a shock absorbing element configured to attenuate a shock resulting from the plunger rod hitting the stopper in response to the actuation of the plunger rod, wherein the shock absorbing element is either (i) embodied in the plunger rod of claim 16, or (ii) embodied as a resilient member mounted to or integrated with the stopper.
